(19) United States
(12) Patent Application Publication
(10) Pub. No.: US 2020/0058209 A1
(43) Pub. Date: Feb. 20, 2020

(54) SYSTEM AND METHOD FOR AUTOMATED HEALTH MONITORING

(71) Applicant: Ranjan Narayanaswamy SREEDHARA, Bangalore (IN)

(72) Inventor: Ranjan Narayanaswamy SREEDHARA, Bangalore (IN)

(21) Appl. No.: 16/346,376

(22) PCT Filed: Nov. 13, 2017
(86) PCT No.: PCT/IN2017/050528

§ 371 Date: Apr. 30, 2019

(30) Foreign Application Priority Data
Nov. 13, 2016 (IN) 201641034922

(51) Int. Cl. G08B 21/18; A61B 5/00; G16H 40/67; G16H 50/20; G08B 25/10

(52) U.S. Cl. CPC G08B 21/182; A61B 5/746; A61B 5/0022; A61B 5/7221; A61B 2562/06; A61B 5/6802; G16H 40/67; G16H 50/20; G08B 25/10; A61B 5/7275

(57) ABSTRACT

A system and method for automated health monitoring are described herein. The method comprises receiving data pertaining to vital health parameters of the users in real time using a wearable device and processing the data at pre-determined time intervals to identify one or more health indicators associated with the profile of the user. The method further comprises notifying the user and/or one or more users related to the user of the wearable device, the health indicators and precautionary alert messages at regular time intervals. The system and methods of the present disclosure also enable emergency medical care to be provided to the user in an efficient manner.

### FIELD OF INVENTION

The present disclosure relates generally to system and methods for automated health monitoring and more particularly to remote health monitoring and analysis of vital health parameters.

### BACKGROUND

Telemedicine is an emerging field that generally relates to medical care access for consumers and health professionals via telecommunications technologies. One application of telemedicine involves the remote monitoring of vital health statistics of a user and transmitting the statistics to a healthcare facility such as a hospital or medical clinic. For example, a user may measure blood pressure or blood glucose levels and transmit the measurements to their healthcare provider so that the user can be monitored more frequently and without physical visits to an office or healthcare facility. Additionally, devices are known that are used by the user and healthcare provider to monitor a variety of vital health statistics, however, the devices can be relatively expensive, large, and heavy.

For example, U.S. Pat. No. 5,997,476 to Brown discloses a networked system for interactive communication and remote monitoring of users, wherein a monitoring device is provided that produces measurements of physiological conditions of the user, such as blood glucose, and records the measurements for transmission from a remotely programmable apparatus to a system server. The remotely programmable apparatus in one form is a personal computer or remote terminal connected to the server via a wide area network such as the Internet. Unfortunately, the system also requires the user to answer a variety of questions through a user interface on a computer, which are transmitted from the system server, which may not be practical in certain situations, e.g. a medical emergency.

A further known telemedicine device is disclosed in U.S. Pat. No. 6,113,540 to Iliff, wherein both diagnostic and treatment advice is provided when a user (user) accesses a system over a telephone network. Software algorithms provide diagnostic and treatment information based on inputs (complaints) from the user. However, the system of Iliff does not include a means for measuring vital health statistics and transmitting the statistics to a remote location in real time. Further, the system of Iliff is relatively large and heavy and is not designed for a mobile user.

Although known telemedicine devices that transmit vital health statistics to a remote location are effective in measuring and transmitting the appropriate information, no device has yet been developed for efficient use on a mobile platform. For example, cases of in-flight medical emergencies sometimes occur on commercial flights, wherein a passenger or crew member is critically ill and an emergency treatment team or center is waiting on the ground for the arrival of the flight in order to treat the user. Unfortunately, use of telemedicine devices of the known art would not be possible due to the lack of a communications medium onboard the flight. Further, the many known telemedicine devices would be cost, space, and weight prohibitive for use on an aircraft.

Accordingly, there remains a need in the art for a system and method that can monitor the vital health parameters of a subject and efficiently implementing actions in response to the reported vital health parameters. A further need exists for a system that is relatively compact, lightweight and inexpensive, and which is compatible with commonly used computing devices such as laptop computers and personal digital assistants (PDAs).

### SUMMARY

This summary is provided to introduce a selection of concepts in a simple manner that are further described in the detailed description of the disclosure. This summary is not intended to identify key or essential inventive concepts of the subject matter nor is it intended for determining the scope of the disclosure.

In one embodiment, a vital heath monitoring and alert system is disclosed. The system comprises a plurality of sensors communicatively connected to a remote server over a bi-directional communication network. In one embodiment, the plurality of sensors housed in a wearable device are configured to measure the one or more data associated with one or more vital health parameters of a subject in proximal contact with the plurality of sensors.

The data received from the sensors is analysed at the remote server, the analysis comprising at least processing the data to remove noise and quantifying the data to calculate vital health parameters. The calculated values are then processed by a pattern generator module to determine optimal vital health parameters of the subject.

Further, the pattern generated is processed by a prediction module to predict any deviations from a pre-defined threshold for each of the one or more vital health parameters. The output is then selectively communicated to the one or more pre-authorized users over the network. The output may include an alert message.

Several other embodiments, methods for implementing the same and advantages of the present disclosure will be apparent from a reading of the following description and a review of the associated drawings.

### BRIEF DESCRIPTION OF DRAWINGS

The disclosure will be described and explained with additional specificity and detail with the accompanying figures in which:

FIG. 1 is a view of an inventive system for automated monitoring of vital health parameters of a user of a wearable device in accordance with embodiments of the present disclosure.

FIG. 2 illustrates the components of the remote server in communication with the wearable device of the user.

FIG. 3 illustrates a method for automated monitoring of vital health parameters of a user of a wearable device in accordance with embodiments of the present disclosure.

FIG. 4 illustrates an exemplary embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the invention as would normally occur to those skilled in the art are to be construed as being within the scope of the present disclosure.

It will be understood by those skilled in the art that the foregoing general description and the following detailed description are exemplary and explanatory of the disclosure and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may all, but not necessarily do, refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the meaning as commonly understood by one of ordinary skill in the art to which this disclosure belongs. The systems, methods, and examples provided herein are only illustrative and not intended to be limiting.

Embodiments of the present disclosure will be described below in detail with reference to the accompanying figures.

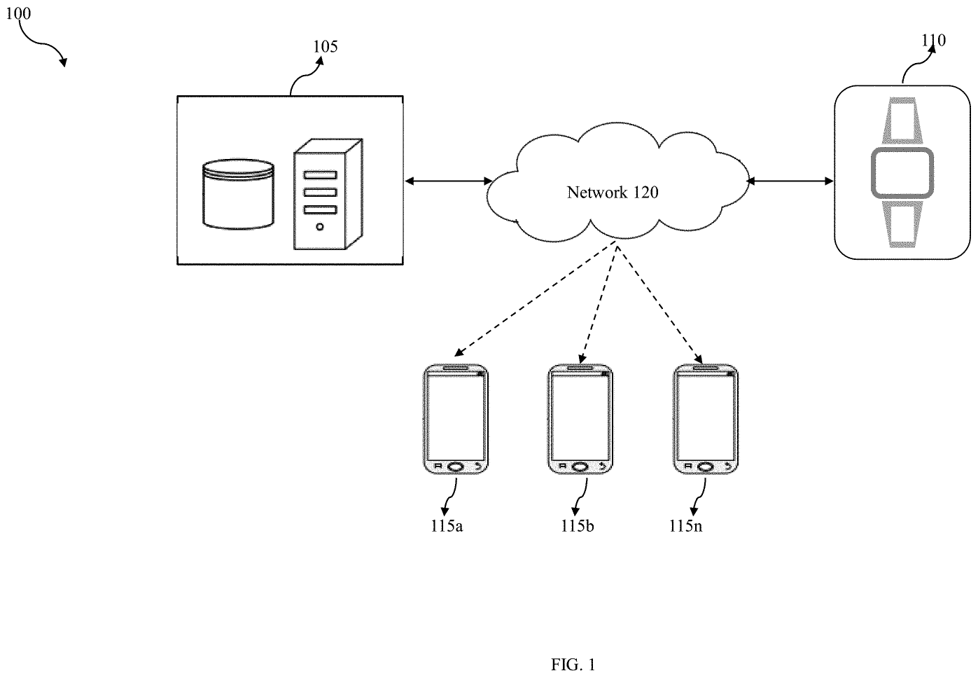

FIG. 1 illustrates a vital health monitoring and alert system 100 . The system 100 includes a remote server 105, a wearable device 110 and one or more user devices 115a to 115n communicating over a network 120.

The network 120 may be the Internet or Wide Area Network (WAN) or other like network that covers a broad area, such as a personal area network (PAN), local area network (LAN), campus area network (CAN), metropolitan area network (MAN), a virtual local area network, Bluetooth communication protocol or other like network capable of physically connecting computers and other devices.

Further, in accordance with the present disclosure, the user device 115a to 115n may include one of a smartphone, a notebook computer, a personal data assistant (PDA) and the like having optionally positioning capability (GPS), capable of connecting to the internet and having other communication capabilities.

The remote server 105 includes one or more processing modules configured to perform one or more actions in accordance with embodiments of the present disclosure. The functionality is shown organized in discrete functional modules for purposes of explaining the software and hardware by which the remote server 115 may be implemented. It has to be noted that such hardware and software may be conjoined, subdivided, replicated, or otherwise differently arranged relative to the illustrated functional modules. Further, the remote server 115 may be implemented on one or more computing devices based on the requirements, such as based on the data set to be processed, time and cost and other constraints.

In an embodiment of the present disclosure, the functionalities of the remote server 105 and the wearable device 110 are distributed. The wearable device 110 is communicatively connected to the remote server 105 over the bi-direction communication network 120. The wearable device 110 is worn by the subject (not shown) for measuring one or more vital health parameters. The wearable device 110 houses a plurality of sensors including but not limited to a temperature sensor, a pressure sensor, a non-invasive blood glucose sensor, an oxygen saturation sensor and leads for heart rate monitoring. In another embodiment, one or more wearable devices 110 each housing a different sensor may be worn by the subject for measuring vital health parameters from the most proximal regions. In yet another embodiment, the wearable device 110 may further comprise a storage means for storing the data monitored by the sensors transiently before communicating the same to the remote server 105.

The term ‘user’ and ‘subject’ are used interchangeably and refer to the user wearing the wearable device 110 for monitoring the vital health parameters of the user. In one embodiment, the wearable device 110 may have additional functionality such as a GPS for recording location, a communication module for communicating with the remote server 105 and the user device 115, an audio function, a tactile function, a power module and the like. Plurality of such wearable device 110 may be worn by the subject for example on the wrist, arm, chest, neck etc.

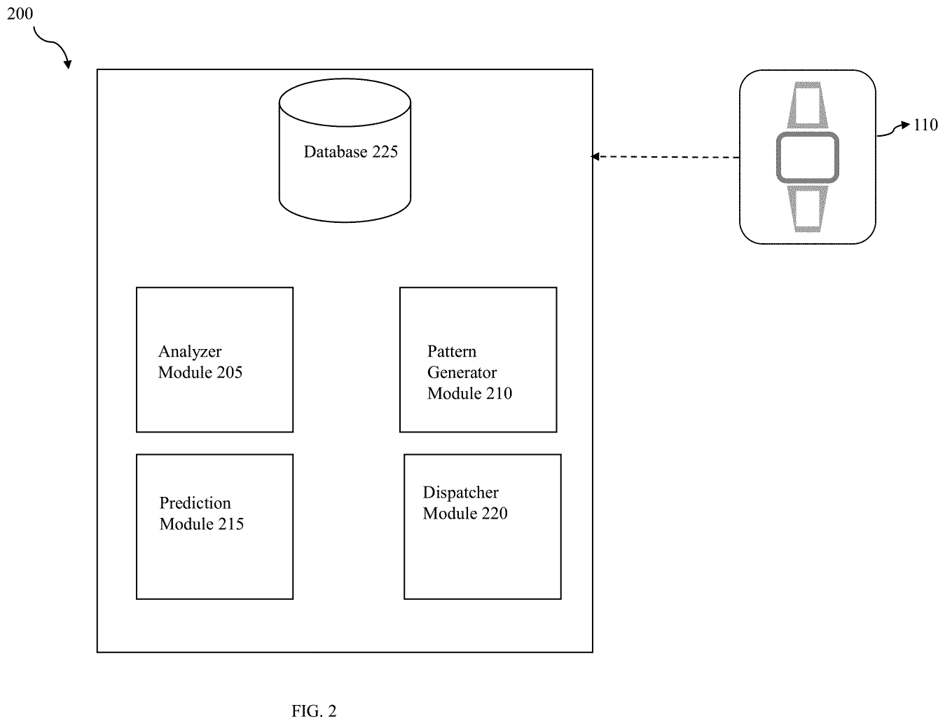

Now referring to FIG. 2, the one or more processing modules of the remote server 105 are described in detail. The one or more processing modules comprise a receiver module (not shown), an analyser module 205, a pattern generator module 210, a prediction module 215 and a dispatcher module 220. Further, the remote server 105 comprises a database 225.

The receiver module is configured to receive the measured data from the one or more sensors of the wearable device 110. In one embodiment, the receiver module is implemented on a pull mechanism such that the receiver module sends a request to the wearable device 110 over the communication network 120 to receive the data. In another embodiment, the receiver module receives the data from the wearable device 110 asynchronously and timestamps the data packets. In yet another embodiment, the receiver module is configured to receive the data from the wearable device 110 continuously or in batches.

The timestamped data received from the wearable device 110 is stored in the database 225 against a unique ID associated with the subject. The unique ID is created by the remote server 105 through an interface on the user device associated with the subject upon receiving a request from the subject for using the vital health monitoring system of the present disclosure. The request received from the subject comprises the profile information including but not limited to a name, gender, age, location, one or more alternate contacts, one or more emergency contacts, personal physician information, body mass index, weight, height, allergy information and the like.

In an embodiment of the present disclosure, the profile information received from the subject is further validated by the system 100 using third party APIs and the like provided by a centralised user health databases maintained by hospitals, state facilities and the like.

The analyser module 205 is configured to quantify the data received by the receiver module. The data received from the one or more sensors housed in the wearable device 110 is processed by the analyser module 205 to generate a value for each of the one or more measured vital health parameters for every instance. For example, the electrical activity measured by the leads on the wearable device 110 are processed by the analyser module 205 to calculate the heart rate or pulse of the subject. In one embodiment, the analyser module 205 implements algorithms to correct the anomalies in the data received from the one or more sensors. For example, the temperature data received from the sensor may be adjusted by the analyser module 205 using contextual information such as the room temperature at the location of the subject.

Similarly data indicative of various physiological parameters of an individual, such as the individual's heart rate, pulse rate, beat-to-beat heart variability, EKG or ECG, respiration rate, skin temperature, core body temperature, heat flow off the body, galvanic skin response or GSR, EMG, EEG, EOG, blood pressure, body fat, hydration level, activity level, oxygen consumption, glucose or blood sugar level, body position, pressure on muscles or bones, and UV radiation exposure and absorption. In certain cases, the data indicative of the various physiological parameters is the signal or signals themselves generated by the one or more sensors and in certain other cases the data is calculated by the microprocessor based on the signal or signals generated by the one or more sensors

In accordance with embodiments of the present disclosure, data relating to the physiological state, the lifestyle and certain contextual parameters of an individual is collected and transmitted, either subsequently or in real-time, to a site, preferably remote from the individual, where it is stored for later manipulation and presentation to a recipient, preferably over an electronic network such as the Internet. Contextual parameters as used herein means parameters relating to the environment, surroundings and location of the individual, including, but not limited to, air quality, sound quality, ambient temperature, global positioning and the like. Referring to FIG. 1, located at user location is the wearable device 110 adapted to be placed in proximity with at least a portion of the human body. Wearable device 110 is preferably worn by an individual user on his or her body, for example as part of a garment such as a form fitting shirt, or as part of an arm band or the like. Wearable device 110, includes one or more sensors, which are adapted to generate signals in response to physiological characteristics of an individual, and a microprocessor. Proximity as used herein means that the sensors of wearable device 110 are separated from the individual's body by a material or the like, or a distance such that the capabilities of the sensors are not impeded.

The pattern generator module 210 is configured to implement one or more algorithms to group the processed data into one or more vital health indicator groups. The health indicator groups, for example, comprise of historical data associated with one or more vital health parameters of the subject and the vital health parameters of plurality of users belonging to the same age group, same locality, same gender and the like. In one exemplary embodiment, the pattern generator module 210 collects the blood pressure data of the subject from the analyser module 205 and estimates the average systolic and diastolic blood pressure of the subject. Further, the pattern generator module 205 compares the average blood pressure of the subject with plurality of groups to identify indicators associated with health of the user. For example, if the average blood pressure of the user aged 45 years is 140/90 units and the user profile shows past history of high cholesterol levels, the pattern generator module 210 stores the patterns for each of the one or more vital health parameters of the subject in the database 225. Further, the pattern generator module may be configured to track and update the patterns at pre-defined time intervals.

The prediction module 215 is configured to learn from the patterns generated for each of the one or more subjects and perform a classification for each of the one or more vital health parameters of the subject. The prediction module 215 may implement algorithms based on Markov models, linear regression models, cluster algorithms and the like. Specifically, the prediction module 215 compares and/or correlates the patterns generated by the pattern generator module 210 with a sample data for each of the one or more vital health parameters to predict a deviation from the optimal values for the one or more vital health parameters. The sample data is, for example, provided as an input to the prediction module 215 from a crowd sourced data, a clinical data and the like. The predictions may further be correlated with clinical symptoms and stored in the database 225 for further assessment by authorized users. In an embodiment, a threshold value for each of the one or more vital health parameters is determined from the sample data and the patterns generated for the subject are compared with the said threshold value in order to determine the vital health status of the user.

The dispatcher module 220 is now configured to dispatch the vital health status of the subject to the one or more authorized users associated with the user operating the user device 115a-115n. In one embodiment, the vital health status comprises of the predictions for each of the one or more vital health parameters, a list of clinical symptoms, patterns associated with the one or more vital health parameters and the like. Further, the vital health status also comprises the pre-defined range of vital health parameters, a significance of the value of the one or more vital health parameters measured and a comparison with the previously recorded value for the one or more vital health parameters.

Further, the dispatcher module 220 is configured to raise an alert via SMS, IVR or other communication means to the one or more authorized users operating the user device 115a-115n including but not limited to the physician of the subject. In one embodiment, the dispatcher module 220 may be configured to communicate the pattern and analysis report at pre-defined time intervals to the user and/or other users associated with the user operating the user device 115a-115n. In yet another embodiment, the dispatcher module 220 may be configured to communicate the vital health status, the predictions and the patterns associated with subject to a medical facility in the vicinity of the subject as well as to the subject itself upon determining a deviation of the vital health parameters of the subject from the optimal threshold. It is to be noted that in the event of the pattern indicating an adverse or abnormal pattern associated with vital health parameters of the user, the dispatcher module 220 may override the default configurations and send the status to the one or more users.

In an embodiment, the dispatcher module may be configured to communicate the patterns for the one or more vital health parameters in response to a scheduled plan generated by the subject using an application on the user device 115 associated with the subject. For example, a subject with history of hypertension symptoms may register with the system 100 for monitoring the blood pressure levels for a period of 30 days with a target of bringing the blood pressure levels down by 20%. For the above stated example, the prediction module first determines a threshold for the user group having historic hypertension system and predicts an optimal plan for bringing down the blood pressure levels within the set goal of 30 days. The dispatcher module 220 may then accordingly communicate the blood pressure values to the subject for the defined period.

In another embodiment, the remote server 200 additionally comprises a location integrator module (not shown) which may be configured to access the current location of the user of the wearable device 110. Further, the location integrator module 225 may pin the user on a pre-loaded location map or by accessing map data from third party sources using application programming interface (APIs), such as Google Maps. In the event of health emergency, as explained in the sections above, the location integrator module 220 may identify the nearest medical facilities or ambulance services and trigger an alert communication providing the current location of the subject.

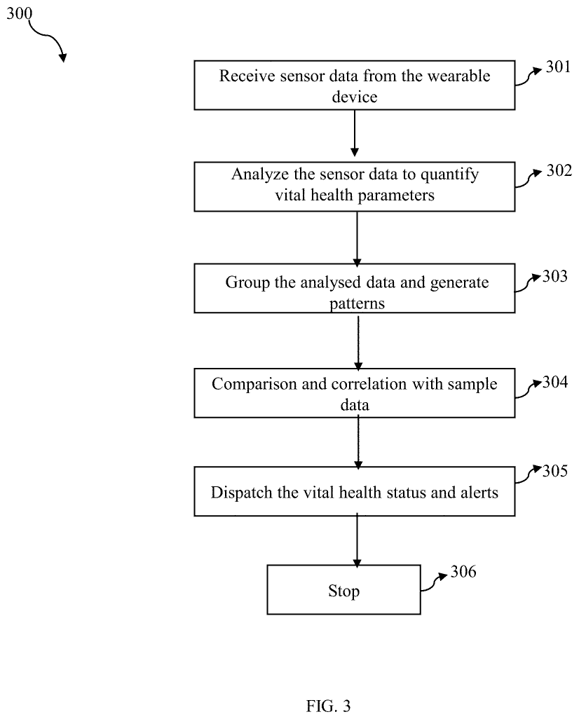

Now referring to FIG. 3, an exemplary method for automated monitoring of vital health parameters associated with a user in accordance with embodiments of the present disclosure is described herein. The method starts with step 301. At step 301, the one or more vital health parameters associated with the user wearing the wearable device 105 is received at the remote server 110. The one or more health parameters may be received in real time or may be configured to be received at pre-determined time intervals. The one or more sensor data include but are not limited to heart rate, temperature, blood pressure, pulse, blood sugar and the like. The sensor data is stored in the database 225 against a unique ID associated with the user.

At step 302, the sensor data is retrieved by the analyzer module 205 for further processing. The analyzer module 205 processes the raw sensor data and removes noise. Further, the analyzer module 205 segregates the raw sensor data into specific vital health parameters and determines the average and minimum, maximum data points.

At step 303, the processed data is received by the pattern generator module 210. The pattern generator module 210 groups the processed data and the user profile information. Further, the pattern generator module 210 compares the processed data with historical user data as well as with user group data to generate patterns and estimate any health indicators associated with vital health parameters of the user.

At step 304, the patterns and one or more vital health indicators are then compared with pre-defined thresholds derived from a sample data by the prediction module 215. The prediction module 215 thus generates a holistic report on the status of the one or more vital health parameters.

At step 305, the status of vital health parameters is transferred to the dispatcher module 215. The dispatcher module 215 then selectively communicates the status and alert message if any along with the patterns to the one or more users pre-authorized by the subject wearing the wearable device 110. In one embodiment, the status and/ or alert is communicated at pre-defined time intervals as configured by the user. In one exemplary embodiments, upon determining that the patterns and/or health indicators are indicative of adverse health conditions, the dispatcher module may override the default settings and send alert messages to the one or more users associated with the user of wearable device 110. In yet another embodiment, the dispatcher module 215 is also configured to send the health indicator to one or more medical facilities and/or hospitals in proximity to the user of the wearable device 110.

The process terminates at step 306.

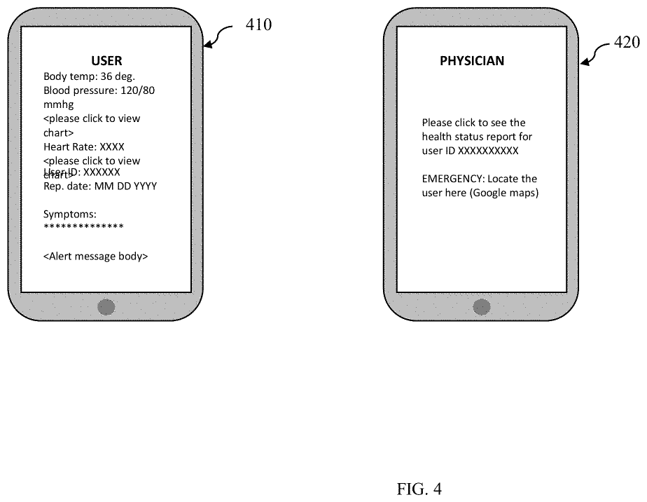

FIG. 4 illustrates an exemplary screen of the user device 410 associated with the subject and the user device 420 operated by the physician of the subject. As can be seen, the dispatcher module 220 of the system communicates customized status reports and/or alert notifications to the users and the notification further includes emergency alerts, if any, for the users.

It is evident from the foregoing description that the various embodiments of the present disclosure are advantageous in monitoring the health conditions of the user in real time thereby enabling users to get immediate healthcare. Further, the system and methods of the present disclosure are advantageous in that the vital health parameters are analyzed over a period of time, which would in turn help the users take preventive care.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

I/We claim:

1. A vital health monitoring and alert system, the system comprising:
one or more sensors in proximal contact to a subject;
a bi-directional communication network for transmitting a data between the one or more sensors and a remote server;
the remote server comprising at least a processor and a memory coupled to the processor, wherein the memory is configured to store:
a receiver module for receiving the data from the one or more sensors, wherein the data is indicative of one or more vital health parameters of the subject measured instantaneously by the one or more sensors;
an analyzer module for processing the data to quantify the one or more vital health parameters of the subject;
a pattern generator module for determining a quality of the one or more vital health parameters of the subject, wherein the quality is an indicator of the vital health of the subject,
a prediction module for performing one of a comparison and correlation of the pattern generated by the pattern generator module, wherein the comparison and correlation is performed with a pre-defined threshold and
a dispatcher module for selectively dispatching the determined one or more health indicators deviating from the pre-defined threshold, to one or more contacts pre-selected by the subject.

2. The system as claimed in claim 1, wherein the one or more sensors are housed in a wearable device to be worn by the subject.

3. The system as claimed in claim 1, wherein the pattern generator module is configured to determine the quality of the one or more vital health parameters of the subject based on at least a historic health pattern of the subject.

4. The system as claimed in claim 1, wherein the pre-defined threshold is determined using a sample of crowd sourced data or a clinical data for the one or more vital heath parameters.

5. The system as claimed in claim I wherein the one or more contacts for communicating the alert are configured in a user application running on a mobile device associated with the subject.

6. The system as claimed in claim 1, wherein the dispatcher module is configured for communicating the alert to the healthcare facility in proximity of the subject upon determining a deviation from the pre-defined threshold of the one or more vital health parameters.

7. A method for vital health monitoring of a subject, the method comprising:
receiving a data from one or more sensors in proximal contact with the subject, wherein the data is indicative of one or more vital health parameters of the subject measured instantaneously by the one or more sensors;
processing the data received from the one or more sensors to quantify one or more vital health parameters of the subject;
determining a quality of the one or more vital health parameters of the subject, wherein the quality is an indicator of the vital health of the subject; and
performing one of a comparison and correlation of the one or more vital health parameters with a predefined threshold to determine the vital heath status of the subject.

8. The method as claimed in claim 7, wherein the method further comprises the steps of communicating the vital health status of the subject to one or more contacts pre-selected by the subject.

9. The method as claimed in claim 7, wherein the processing comprises the steps of combining the data received from the one or more sensors to quantify the one or more vital health parameters.

10. The method as claimed in claim 8, wherein the method comprises the steps of communicating the vital health status of the subject to a healthcare facility in proximity of the subject upon determining a deviation the one or more vital health parameters from the pre-defined threshold.
